(19) United States
(12) Patent Application Publication
(10) Pub. No.: US 2006/0204600 A1
(43) Pub. Date: Sep. 14, 2006

(54) Dragon's blood anti-viral materials and methods

(76) Inventors: Paul Konz, Oviedo, FL (US); Gary Pekoe, Virginia Beach, VA (US); Charles Shaffer, Draper, VA (US); Francois Almeida, Orlando, FL (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 11/078,987

(22) Filed: Mar. 11, 2005

(51) Int. Cl. A61K 36/81

(52) U.S. Cl. 424/769

(57) ABSTRACT

The present invention provides for the administration of compounds, which can be synthesized or can be isolated from a Croton plant species for treating invertebrates or vertebrates, including humans, infected with Poxyiridae or Papillomavirinae. The present invention encompasses methods of using compounds synthesized or isolated from the Croton plant species, singularly or synergistically.

### FIELD OF THE INVENTION

The invention relates to methods and compositions for the treatment or prevention of Poxyiridae and Papillomavirinae viral infections, Coxsackie viral infections, cancers affecting the skin, and/or their symptoms in humans and animals, including variola (smallpox) virus, Molluscum contagiosum virus, and human papilloma virus (hpv).

### BACKGROUND OF THE INVENTION

“Dragon's blood” is a red viscous latex sap or resin dervived from the forest euphorb tree (various trees of the genus Croton, extract also known as Sangre de Drado, Sangre de Grago, Drago, Sangue de Drago, Sangue de Agua). Products derived from the Croton tree species have been traded and used for centuries. These products have included therapeutic treatments, food additives, and natural medicines. Relevant Croton tree species includes the Croton salutaris, Croton gossypifolius, Croton palanostima, Croton erythrochilus, Croton lechleri, and Croton draconoides.

The Poxyiridae include both vertebrate and insect viruses. See generally Moss, B., Chp. 34, Fundamental Virology 3^{rd} ed. (1996). Two of the most notable Poxyiridae are small pox and molluscum. Molluscum contagiosum infection is characterized by small flesh-colored or pink dome-shaped growths that often become red or inflamed. They may appear shiny and have a small indentation in the center. Molluscum are usually found in areas of skin that touch each other such as the folds in the arm or the groin. They are also found in clusters on the chest, abdomen, and buttocks and can involve the face and eyelids. In people with immune system diseases, the molluscum may be very large in size and number, especially on the face. To confirm the diagnosis of molluscum, a physician might scrape some cells from the growth and look at them under a microscope. Prescribed topical creams Retin-A and Aldera have been used to treat molluscum. Moluscum lesions may be removed by freezing or surgery. However, these existing therapies have their limitations and new therapies for molluscum infection are needed.

Smallpox is a human viral disease that spreads by inhalation of air droplets or aerosols. 12 to 14 days after infection, an infected individual usually develops a fever and has severe aching pains and prostration. After an additional 2 to 3 days, a papular rash develops over the face and spreads to the extremities. This rash soon becomes vesicular and later, pustular. The infected individual's fever persists throughout the evolution of the rash and severe pain is associated with the growth and expansion of the pustules. Scabs form over time. These scabs eventually separate, leaving pitted scars. The infected individual usually dies within the second week. Vaccination and patient isolation are currently the only tools against small pox. Vaccination before exposure or within 2 to 3 days after exposure affords almost complete protection against disease. Vaccination as late as 4 to 5 days after exposure may protect against death. However, vaccination is associated with some risk for adverse reactions; the two most serious are postvaccinal encephalitis and progressive vaccinia. Given the lethality of small pox, there has been considerable concern of its use as a biological weapon by terrorists. Accordingly, new therapies for treating small pox are sought.

The Papillomavirinae include both human and animal viruses. See generally Howley, P. M. Chp. 29 in Fields Virology 3^{rd} ed. (1996). Human papillomavirus (HPV) ranks as one of the world's most common causes of sexually transmitted infection (STI). There are high-risk and low-risk types of HPV. High-risk HPV may cause abnormal Pap smear results, and could lead to cancers of the cervix, vulva, vagina, anus, or penis. Low-risk HPV also may cause abnormal Pap results or genital warts. HPV has no known cure. Existing treatments for hpv-caused genital warts include Imiquimod cream, 20 percent podophyllin antimitotic solution, 0.5 percent podofilox solution, 5% 5-fluorouracil cream, and Trichloroacetic acid (TCA). Other treatments include freezing (cryosurgery), burning (electrocautery), laser, and surgery. Alpha interferon injections have also been used, despite the expense of this drug. Accordingly, new treatments for hpv infections are sought.

The coxsackie viruses are part of the enterovirus family of viruses (which also includes echoviruses, polio, and hepatitis A). They live in the human digestive tract and are easily spread from person to person, usually on unwashed hands, contaminated surfaces, and from sneezing and coughing. Infections are frequently marked by fevers, headache, and muscle aches. Coxsackie viruses also cause hand, foot, and mouth disease, characterized by painful red blisters on the throat, tongue, gums, inside of the cheeks, and on the hands and feet. Another coxsackie infection, herpangina, is characterized by red-ringed blisters and ulcers on the tonsils and soft palate. Other coxsackie infections include Pleurodynia, hemorrhagic conjunctivitis, and meningitis. No vaccines exist and few effective therapies exist, other than for moderation of symptoms.

A need exists for new and effective materials and methods for treatment and prevention of viral diseases and lessening their severity.

### SUMMARY OF THE INVENTION

The invention relates to compositions, materials, and methods for treating Papillomavirinae, Poxyiridae, Coxsackie, or Molluscipoxvirus infections and symptoms, including the size and or cellular load of lesions.

In one aspect, the invention encompasses a composition comprising compounds found in the Croton tree species and includes synthesized analogs thereof. The invention also encompasses any isolated or synthesized compound or combinations of compounds found in the Croton tree species used, mixed or in conjunction with any pharmaceutically accepted carrier or administration devices. Compositions of the invention optionally further include one or more pharmaceutically accepted diluents, adjuvants, carriers, excipients, colorants, scenting agents, or the like.

The invention further encompasses compounds and methods for reducing the duration of the symptoms of the Poxyiridae infection, including the symptoms of molluscum contagiosum. The method comprising the step of administrating a therapeutically effective amount of a composition thereof having antiviral activity, so there is a reduction of the duration of the symptoms.

The invention further encompasses compounds and methods for reducing the symptoms of viral infections, including reduction of the number, size, and duration of lesions on the skin, scalp, mouth, nasal cavity, genitals, and other surfaces. The method comprising the step of administrating a therapeutically effective amount of the composition having antiviral activity, causing a reduction of the size lesions.

The invention further encompasses compounds and methods for treating humans, infected with any member of the Poxyiridae Family, including subfamilies and genera discovered, undiscovered, presumed eradicated, created, mutated, or yet to evolve or exist. This invention would include Poxyiridae native to humans and Poxyiridae native to animals but found in human for the purpose of biologic terrorism and those that are not.

In a preferred embodiment, the virus (or viral infection) to be treated is one which causes the symptoms that are clinically verifiable and analogous to the presence of a virus found in the Poxyiridae Family.

In another preferred embodiment, the virus is one which causes the symptoms that are clinically verifiable and analogous to the presence of a virus found in the subfamily Chordopoxyirinae of the Poxyiridae Family.

In another embodiment, the virus is one which causes the symptoms that are clinically verifiable and analogous to the presence of a virus and its species, their serotypes, strains, and isolates, found in the genus Orthopoxvirus in the subfamily Chordopoxyirinae of the Poxyiridae Family, to include what is commonly called “Smallpox.”

In another preferred embodiment, the virus is one which causes the symptoms that are clinically verifiable and analogous to the presence of a virus and its species, their serotypes, strains, and isolates, found in the genus Parapoxvirus in the subfamily Chordopoxyirinae of the Poxyiridae Family.

In another preferred embodiment, the virus is one which causes the symptoms that are clinically verifiable and analogous to the presence of a virus and its species, their serotypes, strains, and isolates, found in the genus Avipoxvirus in the subfamily Chordopoxyirinae of the Poxyiridae Family.

In another preferred embodiment, the virus is one which causes the symptoms that are clinically verifiable and analogous to the presence of a virus and its species, their serotypes, strains, and isolates, found in the genus Capripoxvirus in the subfamily Chordopoxyirinae of the Poxyiridae Family.

In another preferred embodiment, the virus is one which causes the symptoms that are clinically verifiable and analogous to the presence of a virus and its species, their serotypes, strains, and isolates, found in the genus Leporipoxvirus in the subfamily Chordopoxyirinae of the Poxyiridae Family.

In another preferred embodiment, the virus is one which causes the symptoms that are clinically verifiable and analogous to the presence of a virus and its species, their serotypes, strains, and isolates, found in the genus Suipoxvirus in the subfamily Chordopoxyirinae of the Poxyiridae Family.

In another preferred embodiment, the virus is one which causes the symptoms that are clinically verifiable and analogous to the presence of a virus and its species, their serotypes, strains, and isolates, found in the genus Molluscipoxvirus in the subfamily Chordopoxyirinae of the Poxyiridae Family. Accordingly, the invention encompasses compounds and methods for reducing the duration of the symptoms of the Molluscipoxvirus infection, including all the symptoms of Mulluscum contagiosum. The method comprising the step of administrating a therapeutically effective amount of a composition thereof having antiviral activity, so there is a reduction of the duration of the symptoms. The invention further encompasses compounds and methods for reducing the size of the symptoms, to include lesions and tumors. The method comprising the step of administrating a therapeutically effective amount of a composition thereof having antiviral activity, so there is a reduction of the size of the symptoms, to include lesions and tumors. The invention further encompasses a compounds and methods for treating humans, infected with any serotypes, strains, and isolates, found in the genus Molluscipoxvirus, including those discovered, undiscovered, presumed eradicated, created, mutated, or yet to evolve or exist. This invention would include Molluscipoxvirus found in human for the purpose of biologic terrorism and those that are not. In another preferred embodiment, the virus is one which causes the symptoms that are clinically verifiable and analogous to the presence of a virus and its species, their serotypes, strains, and isolates, found in the genus Molluscipoxvirus in the subfamily Chordopoxyirinae of the Poxyiridae Family.

In another preferred embodiment, the virus is one which causes the symptoms that are clinically verifiable and analogous to the presence of a virus found and its species, their serotypes, strains, and isolates, in the genus Yatapoxvirus in the subfamily Chordopoxyirinae of the Poxyiridae Family.

In another preferred embodiment, the virus is one which causes the symptoms that are clinically verifiable and analogous to the presence of a virus found in the subfamily Entomopoxyirinae of the Poxyiridae Family.

In another preferred embodiment, the virus is one which causes the symptoms that are clinically verifiable and analogous to the presence of a virus and its species, their serotypes, strains, and isolates, found in the genus Entomopoxvirus A in the subfamily Entomopoxyirinae of the Poxyiridae Family.

In another preferred embodiment, the virus is one which causes the symptoms that are clinically verifiable and analogous to the presence of a virus and its species, their serotypes, strains, and isolates, found in the genus Entomopoxvirus B in the subfamily Entomopoxyirinae of the Poxyiridae Family.

In another preferred embodiment, the virus is one which causes the symptoms that are clinically verifiable and analogous to the presence of a virus and its species, their serotypes, strains, and isolates, found in the genus Entomopoxvirus C in the subfamily Entomopoxyirinae of the Poxyiridae Family.

In still another embodiment, the virus is a Coxsackie virus or member of the enterovirus family.

The following table provides a non-exclusive and non-limiting summary of some of the viral-mediated conditions in humans treatable according to the invention:

| | | Associated or |
|---|---|---|
| Disease | Description | Causative Virus |
| Boston Exanthem | Mild exanthematous febrile illness | Echovirus 16 |
| | with aseptic meningitis | |
| Bowen's Disease | Squamous cell carcinoma in situ | HPV 16 & 18 |
| Bowenoid Papulosis | Genital papules and plaques | HPV 16 |
| | resembling Bowen's disease | |
| Buschke and Löwenstein | Giant Condyloma | HPV 6 & 11 |
| Butcher's Wart | Warty lesions seen in people who | HPV 7b |
| | handle raw meat | |
| Condyloma Acuminata | Genital Warts | HPV Types 6, 11, |
| | | 16 & 18 |
| Epidermodysplasia | Inherited disorder of HPV | HPV 5, 8, 12, and |
| Verruciformis | infection and SCCs | others |
| Eruptive | | Echovirus 25 & 32 |
| Pseudoangiomatosis | | |
| Erythema Infectiosum | Slapped Cheeks, Reticular | Parvovirus B19 |
| (Fifth Disease) | Exanthem, Anemia | |
| Hand-Foot-and-Mouth | Fever, ulcerovesicular stomatitis, | Coxsackie Virus A-16; |
| Disease | acral erythematous vesicles, | Enterovirus 71 |
| | buttock lesions | |
| Heck's Disease | Small white and pink papules in | HPV 13 & 32 |
| (Focal Epithelial | mouth | |
| Hyperplasia) | | |
| Herpangina | Fever, painful ulcerations in | Coxsackie Viruses |
| | mouth | (A-10) |
| Kaposi's Sarcoma | Vascular Tumor | HHV-8 |
| Measles | Viral Prodrome, then enanthem | Paramyxovirus |
| (Rubeola) | (Koplick spots), then | |
| | maculopapular rash spreading | |
| | craniocaudally | |
| Molluscum Contagiosum | Umbillicated lesions, common in | Poxvirus (DNA) |
| | children and HIV infected | MCV-1 to MCV-4 |
| | individuals | MCV-1 most |
| | | common |
| | | MCV-2 in HIV |
| Myrmecia | Large cup-shaped palmoplantar | HPV 1 |
| | warts | |
| Oral Hairy Leukoplakia | Corrugated white plaque on lateral | EBV |
| | tongue | |
| Orf | Umbilicated nodule after farm | Parapoxvirus |
| | animal exposure | |
| Papular/Purpuric Stocking- | | Parvovirus B19 |
| Glove Syndrome | | |
| Pityriasis Rosea | Usually asymptomatic well- | HHV-7 |
| | known exanthema | |
| Ridged Wart | Wart with preserved | HPV 60 |
| | dermatoglyphics | |
| Rosai-Dorfman | Sinus histiocytosis with massive | HHV-6 |
| | lymphadenopathy | |
| Roseola Infantum | Infants with high fever followed | HHV-6 & 7 |
| (Exanthum Subitum, Sixth | by exanthema | |
| Disease) | | |
| Rubella | Viral prodrome, | Togavirus |
| | lymphadenopathy, pain, | |
| | morbilliform rash, enanthem | |
| | (Forschiemer's spots) | |
| Stucco Keratoses | White hyperkeratotic plaques on | HPV 23b |
| | legs | |
| Variola Major (Smallpox) | 12 day incubation, fever and | Variola (poxvirus) |
| | malaise, then centrifugal | |
| | vesiculopustular rash | |
| Verruca Plana | (Flat Warts) | HPV 3 |
| Verruca Plantaris | (Plantar Warts) | HPV 1 |
| Verruca Vulgaris | Common Warts | HPV 2 |

Among the animal viral diseases treatable according to the invention is Foot-and-mouth disease, an economically devastating disease of cloven-hooved animals and cattle, caused by the Picornaviridae virus; and various animal pox diseases.

Thus, in one aspect, the invention is a therapeutic or prophylactic method of treatment for any of the viral infections described herein, comprising (a) selecting a vertebrate subject in need of prophylaxis or therapy for the virus, e.g., by identifying or diagnosing the presence of a viral infection in a subject or identifying a risk for infection due to infected members of the subject's family, community, etc; and (b) administering to the subject a composition comprising a material from croton species, as described herein in greater detail. Preferred vertebrate subjects include humans, zoo mammals, mammals domesticated as pets, livestock, and racing animals, including but not limited to felines, bovines, canines, equines, porcines, dromedaries, and others; and birds, including but not limited to zoo birds, pets, and farm birds, e.g., eagles, hawks, canaries, parrots, chickens, turkeys, ostrich, and emu.

The invention further encompasses any kit or kits for performing the delivery of the invention compounds and the invention methods to reduce the duration, the size or alleviate symptoms caused directly or indirectly by a Poxyiridae infection. This may or may not include sterile bandages, gauze, tapes, or other applicator devices or administration devices containing the invention compositions and methods.

Another aspect of the invention, related to compositions of the invention, is the use of the components of any composition of the invention for the manufacture of a medicament for treatment or preventions of conditions described herein.

As one aspect, the invention provides methods of preventing, treating, or curing viral infections by Poxyiridae or Papillomavirinae or Coxsackie viruses comprising steps of identifying a human subject having at least one condition selected from the group consisting of: molluscum infection, small pox infection, and human papilloma virus infection; and administering to the human subject a composition comprising Dragon's Blood or a substantial equivalent thereof, wherein the composition is administered in an amount effective to cause a palliation in the symptoms associated with one or more of these conditions, e.g., skin lesions, genital warts, etc.

In a related aspect, the invention provides methods of preventing, treating, slowing the progression of, shrinking, ameliorating the symptoms of, or curing other diseases or conditions which result in skin lesions, including but not limited to herpes simplex virus infection, cutaneous T cell lymphoma, basal cell carcinoma, psoriasis, pressure ulcers, skin and soft tissue bacterial infections, wound healing, and adult-onset acne. An exemplary method comprises steps of identifying a human subject having one or more of these conditions, and administering to the human subject a composition of the invention derived from a croton plant (e.g., a Dragon's Blood composition), wherein the composition is administered in an amount effective to cause a palliation in the symptoms associated with one or more of these conditions, cure the condition, or achieve one of the other therapeutic benefits enumerated above. With respect to lymphomas, carcinomas, or other neoplastic conditions, a reduction in tumor size or a slowing of neoplastic growth is a therapeutically beneficial result.

The identification of appropriate subjects preferably involves selecting individuals who have been medically evaluated and determined to have one or more of the aforementioned list of conditions (or performing a medical examination and diagnosing one or more of the conditions).

The composition(s) is administered in an amount effective to palliate the symptoms of the above-referenced conditions. As indicated herein, any form of administration and pharmaceutical composition is contemplated, preferably topical administration. Those of ordinary skill in the art will readily optimize effective dosages and administration regimens as determined by good medical practice and the clinical condition of the individual subject, taking into account such considerations as therapeutic efficacy, risk of toxicity, and side-effects.

As used herein, the term “antiviral activity” refers to the ability of the composition, method, or treatment regimen to reduce the size, extent, severity, and duration of infections, lesions, or the communicability of the virus (e.g,, Poxyiridae or Papillomavirinae or other virus described herein).

As used herein, the term “administration” refers to the process whereby the composition or method of the invention is introduced to a human or animal, which is the host of a virus in the Poxyiridae Family, and is in need of treatment for the infection. Exemplary routes of administration are described in greater detail below, and include topical administration as a liquid or powder, topical administration on a bandage or other delivery device, and the like.

As used herein, the term “therapeutically effective” refers to when a composition or method of the invention is properly administered in vivo to a vertebrate, such as a bird or mammal, including humans, a measurable beneficial effect occurs. Exemplary beneficial effects are described throughout the application, and include measurable antiviral effects in conditions where viral load can be assayed; a reduction of clinically verifiable and/or patient-reported symptoms, including the reduction, impedance or retardation in the growth of lesions; shrinkage of lesions; reduction in the duration of the symptoms caused by the Poxyiridae or Papillomavirinae virus directly or indirectly; or complete resolution or curing of the viral infection or outbreak.

As yet another aspect of the invention, it is contemplated that the Dragon's Blood or other compositions of the invention are administered to patients in need of treatment in combination with other therapeutics, such as a second agent which is an anti-viral agent. When given in combination with another agent, the amount of Dragon's Blood given may be reduced accordingly. Second agents are administered in an amount determined to be safe and effective at ameliorating human disease.

It is contemplated that the anti-viral agents are administered in the same formulation as Dragon's Blood and given simultaneously. Alternatively, the agents may also be administered in a separate formulation and still be administered concurrently with Dragon's Blood. As used herein, concurrently refers to agents given within 30 minutes of each other. The second agent may also be administered prior to administration of Dragon's Blood. Prior administration refers to administration of the agent within the range of one week prior to Dragon's Blood treatment up to 30 minutes before administration of Dragon's Blood. It is further contemplated that the second agent is administered subsequent to administration of the Dragon's Blood composition. Subsequent administration is meant to describe administration from 30 minutes after Dragon's Blood administration up to one week after Dragon's Blood treatment.

In one embodiment, the Dragon's blood compounds of the present invention may be formulated as a pharmaceutical composition. In a further embodiment, any one of the pharmaceutical compositions of the invention is administered in conjunction with at least one additional antiviral or anticancer agent. Agents contemplated for practicing the invention include, but are not limited to, anti-viral agents, such as those described below. In one aspect, the composition of the invention is administered topically while the second agent is administered orally. In a related aspect, the compound of the invention and the additional agent are both administered topically, either in the same formulation or as separate formulations. It is also contemplated that the additional agent may be an agent useful in treating cancer, such as a chemotherapeutic or radiotherapeutic agent.

Novel formulations that include the croton/dragon's blood material and a second therapeutic agent are themselves aspects of the invention. Such dual agent formulations or kits (when packaged together but not in admixture) optionally further comprise a pharmaceutically acceptable diluent, carrier, stabilizer, or the like, or a delivery agent.

In yet another variation, the invention provides a method of treating an animal having a viral infection or other condition characterized by skin lesions, comprising administering to an animal in need of such treatment a composition of the invention. Any animal that is infected by viruses or suffers from conditions involving skin lesions is suitable for treatment, such as any domestic or zoo animal of economic value, especially mammals and birds. Exemplary animals include dogs and other canines, cats and other felines, cows and other bovines, pigs and other porcines, poultry, primates, pachyderms, equines, and large zoo mammals. The dusting of animals with compositions of the invention, and administration of compositions of the invention through medicated ear tags, collars, and other devices is specifically contemplated.

This summary of the invention is not intended to be limiting or comprehensive, and additional embodiments are described in any drawings and the detailed description, including the examples. All such embodiments are aspects of the invention. Moreover, for the sake of brevity, various details that are applicable to multiple embodiments have not been repeated for every embodiment. Variations reflecting combinations and rearrangements of the embodiments described herein are intended as aspects of the invention. In addition to the foregoing, the invention includes, as an additional aspect, all embodiments of the invention narrower in scope in any way than the variations specifically mentioned above. For example, for aspects described as a genus or range, such as dosages or dosing regimens, every subgenus, subrange or species is specifically contemplated as an embodiment of the invention.

### DETAILED DESCRIPTION

The present application provides materials and methods for treatment of viral diseases, including but not limited to viral diseases that affecting the skin.

Sources of Description of Therapeutic Agents

Therapeutic compositions of the invention comprise material obtained from Croton plants. All parts of the plant may be used. In a preferred embodiment, latex or sap from the plant is used to make compositions of the invention and practice methods of the invention.

The “Dragon's Blood” composition of the present invention may preferably be obtained from any of a number of plant species within the genus Croton (family Euphorbiaceae) that grow in Central and South America, e.g., Amazon region of Peru, Ecuador, Brazil, Dominican Republic, Mexico and Colombia. Other Croton species may also be used. Preferred Croton species include Croton salutaris, Croton gossypifolius, Croton palanostima, Croton erythrochilus, Croton lechleri, Croton urucurana, Croton xalapensis and Croton draconoides. The composition can be derived from one plant, or multiple plants of the same species, or from multiple plants of different species.

Plant specimens of different sizes and ages can be used. In preferred embodiments, the plant specimen is a tree of sufficient age to produce harvestable sap. In some embodiments, the source tree is approximately thirty inches in average diameter and approximately sixty feet high.

Sap (also referred to as resin or latex) from the Croton tree is collected to manufacture the therapeutic composition. On first locates an appropriate tree for harvesting. The typical tree is fast growing, reaching heights of 30-45 feet in 3 years. The sap can be harvested like rubber (at a slower rate). Repeated tapping of the tree can lead to excessive scar damage and fungal infections in the tree. This diminishes productivity. The trees can be harvested at 2-3 years of age. After a tree has fallen, the branches and trunk are cut into smaller segments and the bark is lacerated to allow the resin to escape. These segments are then stacked on collecting sheets to collect the resin as the stack “bleeds.”

The sap can also be collected from living trees. A large collecting sheet, e.g., a 15×15 foot tarp, is attached to the “collecting side” of the tree, e.g., using string, duct tape, or another fastener. The tree is wounded up to about 5 inches deep, and more preferably about 0.5 to 1.5 inches deep, e.g., with a machete, with lengthy slices all up and down the collecting side of the tree (over the tarp), and the tree bleeds (sap falls) for approximately 90 minutes onto the tarp.

Using gloves and a medical facial mask to prevent contamination, the larger debris is removed from the sap. The sap is next transferred from the collecting tarp or container into a sterile (e.g., FDA standard laboratory/medical) container, which is then capped. For example, the tarp is folded and the sap is channeled along the fold into the sterile container. In a preferred variation, the collected sap is micro filtered, e.g., to 30 microns, and bottled for storage and sale. Step filtrations of 100 μm and 30 μm may be used. The product is then packaged or bottled using current Good Manufacturing Practices (GMPs) and Standard Operating Procedures (SOPs) for all processes. During all phases of production and packaging, strict quality control and safety standards are emphasized. Spectrometry and/or other methods may be used to test consistency from multiple collections from a source tree or collect from different trees. In some embodiments, irradiation, chemicals, heat, or other means are used to sterilize the composition. In some embodiments the Dragon's Blood comprises not just the sap from the Croton tree source but also all or part (extract) of the bark, roots, stems, leaves, etc.

Analysis of the sap from an appropriate Croton tree source has revealed a composition comprising compounds such as Cyanidole(flavonolmonomers): (+)-gallocatechin, (+)-catechin, (−)-epicatechin, (−)-epigallocatechin, proanthocyanidin(proanthocyanidindimerB-4, proanthocyanidintrimer, proanthocyanidintetramer, proanthocyanidinheptamer), cedrucine, Daucosterol, Dihydrobenzofuran, Dimethylcedrusine, Isoboldine, korberinA&B, magnoflorine, norisoboldine, procyanidins, resin, tannin, hardwickiicacid, bincatriole, crolechinole, crolechinicacid, coberineA, coberineB, taspine, dihyhrobenzofuranlignans:, -3x,4-O-dimetylocedrusine, -4-O-methylocedrusine, 1,3,5-trimethoxybenzene, 2,4,6-trimethoxyphenol, 4-hydroxyphenethylalcohol, beta-sitosterol, beta-sitosterol-beta-D-, glucopyranoside, beta-Pinene, Betaine, Borneol, Calamenene, Camphene, Cuparophenol, D-Limonen, Dipentene, EO, Eugenol, Euparophenol, alpha-calacorene, alpha-copaene, alpha-pinene, alpha-thujone, beta-Caryophyllene, beta-Elemene, Gamma-Terpinene, Gamma-Terpinole, Lignin, Linalool, Methylthymol, Myrcene, Para-cymene(p-cymene), Pectic-acid, Terpinene-4-ol, Vanillin, piridona, aporfina, quinoleina, and the SP-303 (including:alkaloids, proanthocyanidins (antioxidants), terpines, diterpenes, phenols, tannins, andlignans) simplephenols, phytosterols, and and biologically active alkaloids. Compositions comprising one or more of the compounds may be used in the place of a naturally-obtained Dragon's Blood composition, wherein the artificial compositions approximate the activity or have substantially the same activity of the native composition.

Compositions comprising the following compounds recited in the following may also be used in conjunction with the compositions and therapies of the invention, e.g., when treating Molluscum contagiosum: beta-Pinene (Pinene) (U.S. Pat. Nos. 5,260,342; 5,190,977; 5,126,376; 5,086,076; 4,983,637), betaine (U.S. Pat. Nos. 6,551,795; 6,511,834 6,468,744; 6,376,210; 6,309,823; 6,027,880; 4,374,925; 4,275,149), camphene (U.S. Pat. Nos. 5,260,342; 5,190,977; 5,126,376; 5,086,076; 4,983,637), catechins (U.S. Pat. Nos. 6,369,098; 6,316,465; 6,087,385; 6,028,088), epicatechin (U.S. Pat. Nos. 6,087,385; 6,028,088), epigallocatechin (U.S. Pat. Nos. 6,316,465; 6,087,385; 6,028,088), gallocatechin (U.S. Pat. Nos. 6,087,385; 6,028,088), lignin (U.S. Pat. Nos. 6,132,756; 5,945,116; 4,318,846), linalool (U.S. Pat. Nos. 5,260,342; 5,190,977; 5,126,376; 5,086,076; 4,983,637); myrcene (U.S. Pat. Nos. 5,260,342; 5,190,977; 5,126,376; 5,086,076; 4,983,637), p-cymene (U.S. Pat. Nos. 5,260,342; 5,190,977; 5,126,376; 5,086,076; 4,983,637), proanthocyanidin (proanthocyanidin dimer B-4, proanthocyanidin trimer, proanthocyanidin tetramer, proanthocyanidin heptamer) (U.S. Pat. No. 6,316,465 Ophthalmic uses of PPARgamma agonists & PPARgamma antagonists; 1,3,5-trimethoxybenzene (trimethoxybenzene) (U.S. Pat. Nos. 4,304,787; 4,129,662; 4,126,699 4,126,698 4,126,697 4,126,693 4,055,659). Compositions for the therapies may also comprise proanthocyadidin also known as Pycnogenol® and grape seed extract.

Another analysis of molecules derived from a Croton tree and that may be present in the compositions and therapies of the present invention are set out in the following table:

Presence of Compounds in Croton Lechleri (Euphorbiaceae)

| Compound | Type | Source |
|---|---|---|
| BENZENE,1-3-5 TRIMETHOXY | BENZENOID | BARK |
| | | SAP |
| | | SAP |
| BENZOFURAN-5-YL,2-3-DIHYDRO:2-(3-4- | LIGNAN | SAP |
| DIMETHOXY-PHENYL):7-METHOXY-3- | | |
| METHOXY-CARBONYL-PROPAN-1-OIC ACID | | |
| METHYL ESTER | | |
| BENZOFURAN-5-YL,2-3-DIHYDRO:2-(3-4- | LIGNAN | SAP |
| DIMETHOXY-PHENYL):7-METHOXY-3- | | |
| METHOXY-CARBONYL-PROPEN-1-OIC-ACID | | |
| METHYL ESTER | | |
| BENZOFURAN-5-YL,2-3-DIHYDRO:2-(4- | LIGNAN | SAP |
| HYDROXY-3-METHOXY-PHENYL)-7- | | |
| METHOXY-3-METHOXY-CARBONYL- | | |
| PROPEN-1-OIC ACID METHYL ESTER | | |
| BENZYL ALCOHOL,3-4-DIMETHOXY | BENZENOID | BARK |
| | | SAP |
| BINCATRIOL | DITERPENE | BARK |
| | | SAP |
| BOLDINE,ISO | ISOQUINOLINE | LEAF |
| | ALKALOID | |
| BOLDINE,ISO:NOR: | ISOQUINOLINE | LEAF |
| | ALKALOID | |
| CATECHIN(4-ALPHA-8)-GALLOCATECHIN(4- | FLAVONOID | LATEX (UNSPEC PART) |
| ALPHA-6)-GALLOCATECHIN | | |
| CATECHIN(4-ALPHA-8)-GALLOCATECHIN(4- | FLAVONOID | LATEX (UNSPEC PART) |
| ALPHA-8)-GALLOCATECHIN,(+): | | |
| CATECHIN,(+): | FLAVONOID | LATEX (UNSPEC PART) |
| | | LATEX (UNSPEC PART) |
| | | SAP |
| CATECHIN,EPI:(−): | FLAVONOID | LATEX (UNSPEC PART) |
| | | LATEX (UNSPEC PART) |
| | | SAP |
| CEDRUSIN,3′-4-0-DIMETHYL: | LIGNAN | SAP |
| | | SAP |
| CEDRUSIN,3′-4-0-DIMETHYL:(DL): | LIGNAN | SAP |
| CEDRUSIN,4-0-METHYL: | LIGNAN | SAP |
| CROLECHINIC ACID | DITERPENE | BARK |
| | | SAP |
| | | SAP |
| CROLECHINOL | DITERPENE | SAP |
| | | BARK |
| DAUSCOSTEROL | STEROID | BARK |
| | | SAP |
| | | SAP |
| GALLOCATECHIN(4-ALPHA-6)-EPI- | FLAVONOID | LATEX (UNSPEC PART) |
| GALLOCATECHIN | | |
| GALLOCATECHIN(4-ALPHA-8)-EPI- | FLAVONOID | LATEX (UNSPEC PART) |
| CATECHIN | | |
| GALLOCATECHIN(4-ALPHA-8)- | FLAVONOID | LATEX (UNSPEC PART) |
| GALLOCATECHIN(4-ALPHA-8)-EPI- | | |
| GALLOCATECHIN | | |
| GALLOCATECHIN(4-ALPHA-8)- | FLAVONOID | LATEX (UNSPEC PART) |
| GALLOCATECHIN-(4-ALPHA-8)-EPI- | | |
| GALLOCATECHIN | | |
| GALLOCATECHIN,(+): | FLAVONOID | LATEX (UNSPEC PART) |
| | | SAP |
| GALLOCATECHIN,EPI:(−): | FLAVONOID | LATEX (UNSPEC PART) |
| | | LATEX (UNSPEC PART) |
| | | SAP |
| GLAUCINE | ISOQUINOLINE | LEAF |
| | ALKALOID | |
| HARDWICKIIC ACID | DITERPENE | BARK |
| | | SAP |
| KORBERIN A | DITERPENE | BARK |
| | | SAP |
| KORBERIN B | DITERPENE | BARK |
| | | SAP |
| MAGNOFLORINE | ISOQUINOLINE | LEAF |
| | ALKALOID | |
| PHENETHYL ALCOHOL,4-HYDROXY: | BENZENOID | SAP |
| | | BARK |
| | | SAP |
| PHENETHYL ALCOHOL,4-HYDROXY: | BENZENOID | BARK |
| ACETATE | | |
| PHENOL,2-4-6-TRIMETHOXY: | BENZENOID | SAP |
| | | BARK |
| | | SAP |
| PHENOL,3-4-DIMETHOXY: | BENZENOID | BARK |
| | | SAP |
| PROCYANIDIN B-1 | FLAVONOID | LATEX (UNSPEC PART) |
| | | LATEX (UNSPEC PART) |
| PROCYANIDIN B-2 | FLAVONOID | LATEX (UNSPEC PART) |
| PROCYANIDIN B-4 | FLAVONOID | LATEX (UNSPEC PART) |
| | | SAP |
| SINOACUTINE | ISOQUINOLINE | LEAF |
| | ALKALOID | |
| SITOSTENONE,BETA: | STEROID | BARK |
| | | SAP |
| SITOSTEROL,BETA: | STEROID | SAP |
| | | BARK |
| | | SAP |
| SP-303 | FLAVONOID | LATEX (UNSPEC PART) |
| TASPINE | ALKALOID | SAP |
| | | LATEX (UNSPEC PART) |
| | | SAP |
| | | LATEX (STEM) |
| | | SAP |
| | | LATEX (UNSPEC PART) |
| | | LATEX (UNSPEC PART) |
| | | SAP |
| | | LEAF |
| | | BARK |
| | | SAP |
| THALIPORPHINE | ISOQUINOLINE | LEAF |
| | ALKALOID | |

In some variations, the Dragon's Blood composition further comprises a pharmaceutically acceptable diluent, adjuvant, excipient, or carrier, to facilitate and improve administration to a human subject. Pharmaceutical formulation chemistry is a well developed art, and exemplary formulation materials and methods are discussed herein.

Dragon's Blood compositions may further comprise one or more additional agents to treat or prevent viral infection. Such agents include, but are not limited to vaccinia vaccine, cimetidine, cidofovir, acyclovir, valacyclovir, famciclovir, vidarabine, idoxuridine, trifluridine, ciprofloxin, pencyclovir, ganciclovir, foscarnet, ribavirin, amantadine, rimantadine, cidofovir, oligonucleotides, immune globulins, interferons, tretinoin, ranitidine, famotidine, nizatidine, radiation therapy, bexarotene, denileukin, photopherisis, alemtuzumab, methotrexate, pentostatin, fludarabine, 2-deoxychloroadenosine, doxorubicin, gemcitabine, cyclophosphamide, bone marrow/stem cell transplantation, phototherapy, steroids, and aloe vera. Each of these agents is administered using therapeutic routes, doses, and dosing that has been determined by practitioners and manufacturers, or sometimes lower doses due to synergistic effects of combination therapy.

Poxyirdiae

The compositions of this invention may be used to prevent (propylaxis), treat (ameliorate symptoms, shorten duration of illness, lessen severity), or cure viral infections caused by Poxyiridae. Poxiviridae infections that may be treated by this invention include both vertebrate poxviruses (subfamily Chordopoxyirinae) and insect poxviruses (Entomopoxyirinae, e.g., Entomopoxyirinae A, B, and C). Vertebrate poxviruses include without limitation those members of the genera: Orthopoxvirus (e.g., camelpox, cowpox, ectromelia, monkeypox, racconpox, skunkpox, taterapox, Uasin Gishu, vaccinia, variola (small pox), and volepox), Parapoxvirus (Auzduk disease, chamois, contagious eethyma, orf, pseudocoxpox, parapox of deer, and sealpox); Avipoxvirus (e.g., canarypox, fowlpox, juncopox, pigeonpox, psittacinepox, quailpox, peacockpox, sparrowpox, peguinpox, starlingpox and turkeypox), Capripoxvirus (e.g., goatpox, lumpy skin disease, and sheeppox), Leporipoxvirus (e.g., Hare fibroma, myxoma, rabbit fibroma, squirrel fibroma), Suipoxvirus (e.g., swinepox), and Molluscipoxvirus (e.g., Molluscum contagiosum).

Papillomavirinae

The compositions of this invention may be used to prevent, treat, or cure viral infections caused by Papillomavirinae. Papillomaviminae that can be treated by the composition of the invention include without limitation cattle papillomaviruses, e.g., BPV-1, BPV-2, BPV-4, rabbit papillomaviruses, e.g., CRPV, deer papillomaviruses, e.g., DPV, Mastomys natalensis papillomaviruses, e.g., MnPV, elk papillomaviruses, e.g., EEPV, and human papillomaviruses, e.g., HPV-1, HPV-2, HPV-4, HPV-5, HPV-6, HPV-8, HPV-11, HPV-13, HPV-16, HPV-18, HPV-31, HPV-33, HPV-35, HPV-39, HPV-41, HPV-42, HPV-47, HPV-51, HPV-57, HPV-58, HPV-63, and HPV-65.

Kits and Unit Doses

In related variations of the preceding embodiments, the Dragon's Blood composition may be so arranged, e.g., in a kit or package or unit dose, to permit co-administration with one or more other therapeutic agents, but the Dragon's Blood composition and the agent are not in admixture. In another aspect, the Dragon's Blood composition and the agent are in admixture. In some embodiments, the two components to the kit/unit dose are packaged with instructions for administering the two agents to a human subject for treatment of one of the above-indicated disorders and diseases. The kit may comprise the composition of the invention in combination with a vehicle in a cream or gel base, as a pump-spray, as an aerosol, on an impregnated bandage, a medicated animal ear tag or collar, or in a dropper. The composition of the invention may also be in any one of the above formulations in combination with a second agent, including but not limited to antiviral agents described above, topical steroids, aloe vera and the like cosmeceuticals. In one aspect, the kit includes applicator for administering the composition.

Formulations

Biologically active compounds can be used directly to practice materials and methods of the invention, but in preferred embodiments, the compounds are formulated with pharmaceutically acceptable diluents, adjuvants, excipients, or carriers. The phrase “pharmaceutically or pharmacologically acceptable” refer to molecular entities and compositions that do not produce adverse, allergic, or other untoward reactions when administered to an animal or a human, e.g., topically, transdermally, parenterally, by inhalation spray, vaginally, rectally, by eye drop, or by intracranial injection. (The term parenteral as used herein includes subcutaneous injections, intravenous, intramuscular, intracisternal injection, or infusion techniques. Administration by intravenous, intradermal, intramuscular, intramammary, intraperitoneal, intrathecal, retrobulbar, intrapulmonary injection and/or surgical implantation at a particular site is contemplated as well.) Generally, this will also entail preparing compositions that are essentially free of pyrogens, as well as other impurities that could be harmful to humans or animals. The term “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Preferred topical carriers include creams, salves, foams, lotions, collagen preparations, gels, and ointments.

The Dragon's Blood composition may include acid or base components formulated into pharmaceutically acceptable salts. For example, when an acidic substituent, such as —COOH, is present, the ammonium, sodium, potassium, calcium and the like salts, are contemplated as possible embodiments for administration to a biological host. When a basic group (such as amino or a basic heteroaryl radical, such as pyridyl) is present, then an acidic salt, such as hydrochloride, hydrobromide, acetate, maleate, palmoate, phosphate, methanesulfonate, p-toluenesulfonate, and the like, is contemplated as a possible form for administration to a biological host.

Similarly, where an acid group is present, then pharmaceutically acceptable esters of the compound (e.g., methyl, tert-butyl, pivaloyloxymethyl, succinyl, and the like) are contemplated as possible forms of the compounds, such esters being known in the art for modifying solubility and/or hydrolysis characteristics for use as sustained release or prodrug formulations.

In addition, some components may form solvates with water or common organic solvents. Such solvates are contemplated as well.

Aqueous suspensions may contain the sap composition or active compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the sap or components of it in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Dispersible powders and granules suitable for preparation of an aqueous suspension by the addition of water provide the active composition admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The composition also may be formulated as a dispersable powder for dusting the skin, hair, fur, or feathers of humans or animals. The compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents and scent enhancers.

The compositions may also be in the form of suppositories for rectal administration of the composition. These compositions can be prepared by mixing the composition with a suitable non-irritating excipient that is solid at ordinary temperatures but liquid at the rectal temperature and will therefore melt in the rectum to release the drug. Such materials are cocoa butter and polyethylene glycols, for example.

Therapeutic formulations of the compositions useful for practicing the present invention may be prepared for storage by mixing the selected composition having the desired degree of purity with optional physiologically pharmaceutically-acceptable carriers, excipients, or stabilizers (Remington's Pharmaceutical Sciences, 18th edition, A. R. Gennaro, ed., Mack Publishing Company (1990)) in the form of a lyophilized cake or an aqueous solution. Acceptable carriers, excipients or stabilizers are nontoxic to recipients and may be inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or polyethylene glycol (PEG).

The composition to be used for in vivo administration may be sterile. This is readily accomplished by filtration through sterile filtration membranes, prior to or following lyophilization and reconstitution. The composition for parenteral administration ordinarily will be stored in lyophilized form or in solution.

Therapeutic compositions may be placed into a container having a sterile access port, for example, an intravenous solution bag or vial having a stopper pierceable by a hypodermic injection needle. The route of administration of the composition is in accord with known methods, e.g. topical, or by sustained release systems or implantation device.

Suitable examples of sustained-release preparations include semipermeable polymer matrices in the form of shaped articles, e.g. films, or microcapsules. Sustained release matrices include polyesters, hydrogels, polylactides (U.S. Pat. No. 3,773,919, EP 58,481), copolymers of L-glutamic acid and gamma ethyl-L-glutamate (Sidman, et al., Biopolymers, 22: 547-556 (1983)), poly (2-hydroxyethyl-methacrylate) (Langer, et al., J. Biomed. Mater. Res., 15:167-277 (1981) and Langer, Chem. Tech., 12:98-105 (1982)), ethylene vinyl acetate (Langer, et al., supra) or poly-D(−)-3-hydroxybutyric acid (EP 133,988). Sustained-release compositions also may include liposomes, which can be prepared by any of several methods known in the art (e.g., DE 3,218,121; Epstein, et al., Proc. Natl. Acad. Sci. USA, 82:3688-3692 (1985); Hwang, et al., Proc. Natl. Acad. Sci. USA, 77:4030-4034 (1980); EP 52,322; EP 36,676; EP 88,046; EP 143,949).

In preferred embodiments, the Dragons' Blood composition is a topical composition. In one embodiment, the topical composition is formulatedas a cream, a gel, an emollient, a salve, a liquid spray, an aerosol, or an impregnated bandage. In another aspect, the topical formulation comprises a compound to improve the fragrance of the composition, including but not limited to orange extract or mint extract. In some embodiments, the composition is stored at room temperature in a product bottle, lid firmly closed, for up to one year.

Dose and Dosing

Dragon's blood has proven to be very safe and well tolerated in humans, and the frequency of application or administration to a subject can be adjusted upwardly to achieve a desired therapeutic effect. Subjects experiencing side effects should reduce dosage or discontinue use. Depending on the route of administration, a suitable dose may be calculated according to body weight, body surface areas or organ size. Further refinement of the calculations to determine the appropriate treatment dose is routinely made as part of any medical treatment regimen, especially in view of the dosage information and assays disclosed herein as well as the pharmacokinetic data observed in animals or human clinical trials. Dosage consideration may also be guided by pharmaceutical references, see, e.g., Physician's Desk Reference (Montvale, N.J.), which is incorporated by reference in its entirety.

Dosing in humans may be extrapolated from animal dosages, toxicity studies, and pharmacokinetics, according to standard pharmacological methodologies.

In one embodiment, the liquid Dragon's blood composition is applied via dropper onto lesions and allowed to dry, or rubbed in gently. In a related embodiment, the Dragon's blood composition in cream or gel formulation, is applied on the affected area as a thin drop, and rubbed in gently. In one aspect, it is contemplated that the topical formulation is applied up to 2 times daily to the affected areas.

The therapeutic composition may be administered for any range of time, and if necessary may be administered as long as the symptoms, disease, or disorder remains in the subject. Dosages may be varied during the course of treatment. For example, the dosages may be adjusted if the subject encounters side effects, develops unrelated complications, and/or has a change in the kind, dosage, and/or administration of one or more medications other than those of the combination therapy.

Administration to a subject of the Dragon's Blood therapy may be begun before, during, or after symptoms or evidence of viral infection appear. In some embodiments, the therapy is started as early as immediately, 15 minutes (min)., 30 min., 1 hour(s) (hr.), 1½ hr., 2 hr., 2½ hr., 3 hr., 4 hr., 5 hr., 6 hr., 7 hr., 8 hr., 9 hr., hr., 11 hr., 12 hr., 16 hr., 18 hr., 20 hr., 22 hr., 24 hr., 36 hr., 48 hr., 60 hr., 72 hr., 84 hr., 96, hr., 5 days, 6, days, 10 days, 13 days, 1 week, 2 weeks, three weeks, 4 weeks, 6 weeks, 8 weeks, 10 weeks, 12 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, or more, or an intermediate length of time, following evidence of a viral infection or episode. In some embodiments, the therapy is continued for, hours, days (e.g, 1, 2, 3, 4, 5, 6, 7, 8, 9, 10, or more), 1-10 weeks, 1-12 months, or years, or whenever a persistent virus or its symptoms reappear.

The Dragon's Blood therapy may be administered continuously, every 15 minutes 30 min., 1 hour(s) (hr.), 1½ hr., 2 hr., 2½ hr., 3 hr., 4 hr., 6 hr., 8 hr., 12 hr., 24 hr., 36 hr., 48 hr., 3 days, 4 days, 5 days, 6, days, 1 week, 2 weeks, or frequencies intermediate or less than the foregoing.

In a preferred embodiment, the administration of the Dragon's Blood composition is topical with the composition applied directly on the skin lesions and allowed to dry. This application is performed once, twice, three times, four times, or more daily. Good thorough, gentle washing of the infected skin is generally the first step. Dosage is generally 1-2 drops of the undiluted sap per lesion, twice daily. For example, one drop of the composition is applied to each lesion. Multiple drops are applied to a crop of lesions. The drops are allowed to dry (several minutes) or they are gently rubbed (about 15 seconds) over the lesions until the composition changes to a “creamier” state. It then dries very quickly (several seconds). Rubbing should be gentle to prevent autoinoculation of uninfected tissue.

In some embodiments the composition is first applied to a bandage (e.g., gauze), which is then applied to the lesion. This means of application is particularly useful for difficult regions of the human body (groin, armpit, and eyes). The treated bandage is applied to each lesion. If the bandage is separated from the lesion or if the dressing has been worn for 24 hours, a new, treated bandage should be applied. A new dressing is generally applied every day. In some embodiments, the composition is administered until the symptoms (e.g., skin lesions) disappear, become less pronounced, or problematic side effects occur. A contact rash my develop on the skin of people who are latex intolerant or otherwise intolerant.

Therapy with the Dragon's Blood composition may also be combined with other therapies. For example, when treating a molluscum infection (as well as other infections described above), the therapy may be combined with existing compounds, compositions, and therapies that have been described for molluscum infections. Some of these are described in the following patents, patent application publications, and other references, which are incorporated herein in their entirety: U.S. Pat. Nos. 5,260,342; 5,190,977; 5,126,376; 5,086,076; 4,983,637; 6,551,795; 6,511,834; 6,468,744; 6,376,210; 6,309,823; 6,027,880; 4,374,925; 4,275,149; 4,983,637; 6,369,098; 6,316,465; 6,087,385; 6,028,088; 6,132,756; 5,945,116; 4,318,846; 5,260,342, 6,316,465; 4,304,787; 4,129,662; 4,126,699; 4,126,698; 4,126,697; 4,126,693; 4,055,659.

Veterinary Applications

Materials and methods of the invention can be practiced on animals of economic value, to treat animal viral infections and other skin conditions. Treatment of any domestic pet animal, livestock, zoo animals, circus animals, endangered species, and the like is specifically contemplated.

Poxyiridae virus infection occurs in many animal species important as livestock or pets, causing disease in these animals similar to human disease, which at times can result in serious side effects to the animal or livestock industry. For example, the Cowpox virus which is harbored originally in rodents, can spread to cats, cows, humans, and zoo animals, including large cats and elephants. Transmission to humans traditionally occurs via contact with the infected teats of milking cows. However, infections are currently seen more commonly among domestic cats, from which cowpox can be transmitted to humans. Cowpox infection is a self-limiting disease resulting in vesicles and pustules of the hands in humans and similar areas in animals.

Pseudocowpox virus, the agent of pseudocowpox (Milker's nodules, paravaccinia), causes an epithelial cell infection in handlers of cows. Orf virus infection restuls in painful lesions on the skin of sheep, and goats, and can be serious for lambs whose mouth lesions stop them from feeding. Sheep pox and goat pox may be fatal infections, with visceral as well as dermal lesions. Seal pox may result in a severe skin and flipper infection of captive and wild seals. Myxomatosis infects rabbits, and is typically fatal to the infected animal. Yaba monkey tumor virus causes a histiocytoma, or subcutaneous tumorlike growths, of the head or limbs of primates, especially African monkeys, which are often seen in zoos and are important in biological studies. Tanapox virus causes tanapox, a self-limiting epithelial cell infection in primates. Other virus include pig pox, cat pox, camel pox, Fowl pox, pigeon pox, canary pox, and Ectromelia, which infects mice.

### EXAMPLES

The examples assist in further describing the invention, but are not intended in any way to limit the scope of the invention.

### Example 1

A four-year-old boy was diagnosed by a pediatrician to have a Molluscum contagiosum infection of the Molluscipoxvirus in the subfamily Chordopoxyirinae of the Poxyiridae Family. The boy was reported to have observable skin lesions for one month prior to treatment described herein. Components from the Croton tree species Croton lechleri, and the sap compounds found therein were directly administered to each of the visible skin lesions. One to two drops of the naturally isolated Croton tree sap was applied to the lesion one to two times daily. On the seventh day of topically administering the composition to the lesions, it was observed that more than 10% of the lesions had darkened in color, reduced in size, or developed a scab or the combination of these changes. On the fourteenth day of topical administration to the lesions, more than 80% of the lesions had darkened in color, reduced in size, or developed a scab, the combination of these changes, or were no longer visible. On the twenty-first day of topical application to the lesions, it was observed that more than 95% of the lesions had darkened in color, reduced in size, or developed a scab, the combination of these changes, or were no longer visible. On the twenty-eighth day of therapy, there were no longer any visible skin lesions and therapy was stopped. One year later, the boy was reported to not have had any reoccurrence of lesions or any other symptom related to the Molluscum contagiosum infection of the Molluscipoxvirus in the subfamily Chordopoxyirinae of the Poxyiridae Family.

The American Academy of Dermatology states, “It may take from 6 months up to 5 years for all of the molluscum to go away on their own. They may be more persistent in people with a weakened immune system.” American Academy of Dermatology Public Resource Center Brochure, Hanson et al., Dermatology Online Journal 9(2): 2 The boy in example 1 may have had lesions for an additional time period of four months to four years and ten months without the application of the invention.

### Example 2

A two-year-old girl was diagnosed by a pediatrician to have a Molluscum contagiosum infection of the Molluscipoxvirus in the subfamily Chordopoxyirinae of the Poxyiridae Family. The girl was reported to have observable skin lesions for two months prior to treatment via the administration of the invention. Components from the Croton tree species Croton lechleri, and the sap compounds found therein were directly administered to each of the visible skin lesions. One to two drops of the naturally isolated Croton tree sap was applied to the lesion one to two times daily. On the seventh day of application to the lesions, it was observed that more than 10% of the lesions had darkened in color, reduced in size, developed a scab, or the combination of these changes. On the fourteenth day of application to the lesions, more than 80% of the lesions had darkened in color, reduced in size, or developed a scab, the combination of these changes or were no longer visible. On the twenty-first day of topical application to the lesions, it was observed that more than 95% of the lesions had darkened in color, reduced in size, developed a scab, the combination of these changes or were no longer visible. Daily therapy continued, and on the twenty-eighth day there were no longer any visible skin lesions. Therapy was stopped when all lesions had disappeared. One year later, the girl was reported to not have had any reoccurrence of lesions or any other symptom related to the Molluscum contagiosum infection of the Molluscipoxvirus in the subfamily Chordopoxyirinae of the Poxyiridae Family.

The American Academy of Dermatology states, “It may take from 6 months up to 5 years for all of the molluscum to go away on their own. They may be more persistent in people with a weakened immune system.” The girl in example 2 may have had lesions for an additional time period of three months to four years and nine months without the application of the invention.

### Example 3

A thirty-five year old immune suppressed woman was diagnosed by a dermatologist to have a Molluscum contagiosum infection of the Molluscipoxvirus in the subfamily Chordopoxyirinae of the Poxyiridae Family. The woman was reported to have observable skin lesions for five years prior to treatment via the administration of the invention. Components from the Croton tree species Croton lechleri, and the sap compounds found therein, were directly administered to each of the visible skin lesions and taken orally. One to two drops of the naturally isolated Croton tree sap was applied to the lesion one to two times daily. On the seventh day of this combined oral and topical administration, it was observed that more than 10% of the lesions had darkened in color, reduced in size, or developed a scab or the combination of these changes. On the fourteenth day of oral and topical treatment, more than 80% of the lesions had darkened in color, reduced in size, developed a scab, the combination of these changes, or were no longer visible. On the twenty-first day of topical and oral administration, more than 95% of the lesions had darkened in color, reduced in size, developed a scab, the combination of these changes, or were no longer visible. On the twenty-eighth day of topical and oral treatment, there were no longer any visible skin lesions. Therapy was stopped when all lesions had disappeared. Six months later, the immune suppressed woman was reported to not have had any reoccurrence of lesions or any other symptom related to the Molluscum contagiosum infection of the Molluscipoxvirus in the subfamily Chordopoxyirinae of the Poxyiridae Family.

The American Academy of Dermatology states “It may take from 6 months up to 5 years for all of the molluscum to go away on their own. They may be more persistent in people with a weakened immune system.” The woman in Example 4 may have had lesions for an undeterminable amount of time without the application of the invention, since she is immune suppressed.

### Example 4

A twenty-six year old man was diagnosed by a dermatologist to have a Molluscum contagiosum infection of the Molluscipoxvirus in the subfamily Chordopoxyirinae of the Poxyiridae Family. The man was reported to have observable skin lesions for two weeks prior to treatment via the administration of the invention. Components from the Croton tree species Croton draconoides, and the sap compounds found therein, were directly administered to each of the visible skin lesions and taken orally. One to two drops of the naturally isolated Croton tree sap was applied to the lesion one to two times daily. On the seventh day of topically and orally administering the composition, it was observed that more than 10% of the lesions had darkened in color, reduced in size, developed a scab, or the combination of these changes. On the fourteenth day of topical and oral administration more than 80% of the lesions had darkened in color, reduced in size, developed a scab, the combination of these changes, or were no longer visible. On the twenty-first day of topical and oral administration, more than 95% of the lesions had darkened in color, reduced in size, developed a scab, the combination of these changes, or were no longer visible. On the twenty-eighth day of therapy, there were no longer any visible skin lesions. Therapy was stopped when all lesions had disappeared. Three months later, the man was reported to not have had any reoccurrence of lesions or any other symptom related to the Molluscum contagiosum infection of the Molluscipoxvirus in the subfamily Chordopoxyirinae of the Poxyiridae Family.

The American Academy of Dermatology states, “It may take from 6 months up to 5 years for all of the molluscum to go away on their own. They may be more persistent in people with a weakened immune system.” The man in example 5 may have had lesions for an additional time period of five months to four years and eleven months without the application of the invention.

### Example 5

A 55 year old Clinical Psychologist was suffering from recurrent outbreaks of Coxsackie A19 virus, as diagnosed by her internist.

An amount of 5 drops of Sangre De Drago was applied to a 0.25″×2″ gauze strip. The strip was placed between the subject's bottom lip and gum with the Sangre de Drago directed to the lesions in her mouth. Administration was preformed each evening and morning for 10 days.

On the beginning of the 5th day, the subject reported complete resolution of the virus and has had no recurring out breaks, at which time she ceased the treatments with the Sangre De Drago.

A follow up testing for Coxsackie A19 virus was preformed 2 months later, and no evidence of the virus was detected.

### Example 6

To determine the efficacy of dragon's blood on Molluscum contagiosum infection of the Molluscipoxvirus (MCV) and other viral infections of the Poxyiridae or papilloma viridae family, open label and/or double blind studies of Dragon's Blood and other topical or oral therapies are undertaken.

For example, patients reported to have observable skin lesions for two weeks prior to treatment are divided into treatment groups and administered a composition of the invention, such as a topical application of Dragon's Blood, any other substances used to treat topical lesions, such as imiquimod used to treat MCV, and a control group receiving either no treatment or a placebo treatment. In another variation, an experimental group receives both the Dragon's Blood and a second therapeutic agent.

With the experimental group, a composition of the invention comprising components from a Croton tree species, including Croton lechleri and Croton draconoides, such as the sap compounds found therein, are directly administered (1 to 2 drops, 2× daily) to each of the visible skin lesions. The other topical or oral composition is administered in one group according to standard protocol for that particular composition, such as once daily for four weeks, or in another treatment group, according to the same regimen as the Dragon's Blood composition. For placebo, the same dosing regimen as the composition of the invention is employed.

Beginning on about the seventh day of topical administration, the lesions are observed for reduction in number, in size, change in appearance (e.g. darkening, drying), or the combination of these changes. Patients are interviewed and examined to determine if symptoms (e.g., pain, itching, swelling, fever) are reduced and to monitor for adverse sick effects. Treatment continues and results monitored at least weekly e.g., at days 14, 21, and 28. Treatment may continue because, depending on the nature of the condition being treated, additional time may be required for efficiency to be established compared to controls. Patients are monitored for at least three months after cessation of treatment for return of lesions. Lesions of patients receiving the Dragon's Blood composition, other topical compositions, and the control groups are compared by length of time needed to clear the lesions, percent of lesions remaining at a given timepoint, or average percent reduction in lesion size at a given timepoint.

In addition to treatment of skin lesions associated with pox virus infection, it is further contemplated that the Dragon's Blood compositions are useful in the treatment of other diseases or conditions resulting in lesions on the skin. Dragon's blood may be administered to subjects as above and compared with therapy known in the art to treat such conditions. Table 1 describes the condition to be treated, the current treatment for such condition and an exemplary clinical trial formed to determine the efficacy of Dragon's Blood in treating the condition.

In one aspect, the comparisons are performed as an open label trial, wherein both the doctors and patients are aware of what treatment is being given. Note that an open label trial can be randomized, or non-randomized, as long as the patients and doctors know what treatment has been assigned. In a related aspect, the studies are also performed as a parallel group study, wherein the results of a treatment on two separate groups of patients are compared.

TABLE 1

| | | | Minimum No. | | |
|---|---|---|---|---|---|
| Indication | Current Treatment | Treatment Scheme | of Subjects | Length of Study | |
| Herpes simplex, | acyclovir (Zovirax), valacyclovir | parallel-group, open- | 20 | 5 | days |
| HSV | (Valtrex), famciclovir (Famvir) | label | | | |
| Cutaneous T | psoralen and ultraviolet A light (PUVA) | open-label | 10 | 12 | weeks |
| Cell lymphoma | therapy, topical chemo or sterioids | | | | |
| Basel Cell | Surgery | open-label | 10 | 12 | weeks |
| Psoriasis | Corticosteroids, numerous others | parallel-group, open- | 20 | 12 | weeks |
| | | label | | | |
| Atopic | numerous topical therapies | parallel-group, open- | 20 | 8 | weeks |
| Dermatitis | | label | | | |
| Common warts | salicylic acid | parallel-group, open- | 20 | 8 | weeks |
| | | label | | | |
| Poison ivy | calamine lotion | parallel-group, open- | 20 | 8 | weeks |
| | | label | | | |
| HPV/Genital | Aldara ™ (imiquimod) Cream, 5%. | parallel-group, open- | 20 | 8 | weeks |
| warts | | label | | | |
| MCV | Aldara ™ (imiquimod) Cream, 5%. | parallel-group, open- | 20 | 8 | weeks |
| | | label | | | |

It is contemplated that the length of time for the study, or the number of participants in the study may be altered by the treating physician. In the above studies, the Dragon's Blood composition is administered topically as described previously, 1-2 drops on the lesion, 2× daily. In groups receiving other topical treatment, it is contemplated that the topical treatment may be administered either as prescribed by the manufacturer or over the same regimen as the Dragon's Blood composition.

For the conditions listed in Table 1, the effects of Dragon's Blood are compared to untreated controls (if appropriate) or a treatment standard in the art for the condition being treated, and the change in skin lesion recorded. For example, in comparison of Dragon's Blood and topical corticosteroids in the treatment of psoriasis, the area of the affected skin lesion is compared between patients receiving Dragon's Blood and those receiving corticosteroid treatment. A decrease in lesion size after treatment with a Dragon's Blood composition indicates that Dragon's blood is an effective treatment for individuals having psoriasis. The same type of assessment is made in patients having cutaneous T cell lymphoma, warts, atopic dermatitis, or any other condition listed in Table 1. An improvement in the symptoms of the condition after treatment with a Dragon's Blood composition of the invention indicates that Dragon's blood is an effective treatment for the skin condition.

### Example 7A

The following animal study is useful for demonstrating efficiency of the invention in a veterinarian context and also for providing preclinical evidence of efficiency relevant to human thereapy with Dragon's Blood alone or in combination with a second anti-viral agent.

Monkeypox produces a disease in monkeys is very similar to smallpox infection in humans. Often, clinically, monkeypox infection cannot be distinguished from smallpox. To assess the efficacy of Dragon's Blood on monkey pox and small pox infection, an animal model of monkeypox infection is used. For example, the Cynomolgus Monkey Model described in Jahrling et al., (Proc Natl Acad Sci USA. 101:15196-200, 2004), incorporated hereby reference, is used to assess the efficacy of Dragon's Blood in an animal model of smallpox.

Cynomolgus macaques are exposed to one or several variola strains through aerosol and/or intravenous routes. Aerosol infection of cynomolgus monkeys with monkeypox produces a lethal fibrinonecrotic bronchopneumonia, while a lesional model of disease is induced by intravenous injection of monkeypox (see abstract for Cidofovir Treatment of Smallpox and Monkeypox in the Cynomolgus Monkey Model J. W. Huggins, S. H. Zwiers, R. O. Baker, L. E. Hensley, T. Larsen, M. J. Martinez, P. J. Jahrling; USAMRIID, Fort Detrick, Md.).

Once lesions have appeared on the animals, the affected subjects are treated with Dragon's Blood as described above, 1 or 2 drops on the lesion 2 times daily. The lesion sizes and numbers are then assessed as previously described.

The affected animals may also be treated with combination therapies, wherein they receive topical treatment with Dragon's Blood in combination with a second anti-viral agent, such as cidofovir, cyclic HPMPC, or ribavirin (Baker et al. Antiviral Res. 57:13-23, 2003). The animals are then assessed for lesion size as well as viral load, as described in (abstract for Real-Time TAQMAN®-MGB PCR Assay of Smallpox, Monkeypox, and Cowpox Genomes in Blood and Tissues from Experimentally Infected Animals S. H. Zwiers, D. Miller, R. O. Baker, D. Kulesh, P. B. Jahrling, J. W. Huggins; USAMRIID, Fort Detrick, Md.).

Viral particles are measured using real-time PCR adapted from methods described in Kulesh et al., (Nature 84:1200-1208, 2004, for detecting MPXV), Ibrahim et al., (J Clin Microbiol 41:3835-3839, 2003, for detecting smallpox virus) and Espy et al., (J Clin Microbiol 40:1985-88, 2002, for detecting orthopoxvirus). Briefly, blood of experimentally infected primates is extracted with the Qiagen QIAamp DNA Mini Kit and incubated in buffer to inactivate the virus. In one aspect, quantitative PCR is performed using TAQMAN®-MGB and a panorthopox primer/probe set directed against the viral hemagglutinin gene. A reduction in virus levels in the dragon's blood treated animals indicates that the composition has an effect on viral replication or viral killing.

### Example 7B

Animal models are also useful to determine the effects of Dragon's Blood on other poxvirus infections such as vaccinia virus or cowpox. For example, Balb/c mice (Smee et al., Antivir Chem Chemother. 12:71-6, 2001, or hairless mice (Smee et al., J Infect Dis. 190:1132-9, 2004) are infected intranasally with vaccinia or cowpox virus and examined daily for skin lesion development. Once lesions appear, animals are treated with 1-2 drops of Dragon's Blood or control substance 2× daily and monitored for improvement in the lesion and reduction in iral load.

### Example 7C

Using an animal model of human papilloma virus infection, the effects of dragon's blood compositions in healing HPV papillomas or warts is assessed. For example, immunocompromised SCID mice receive xenografts of HPV infected foreskin cells as described in Bonnez et al., (J Virol 72:5256-61, 1998). The mice accept the graft and exhibit symptoms of HPV infection similar to humans, such as skin papillomas. The papillomas are treated with the compositions of the invention as described above, and the lesion size assessed for reduction in size, change in color, and a combination of the two symptoms.

A reduction in lesion size after treatment with the compositions of the invention indicates that Dragon's Blood compositions are also an effective treatment for papilloma virus infections

### Example 8

The ability of Dragon's Blood to directly affect the virus replication and activity is also measured using in vitro assays.

The effects of Dragon's Blood on viral killing can be assessed as described in Smee et al., Antimicrobial Agents and Chemotherapy, 46:1329-35, 2002. Camelpox (Somalia strain), cowpox (Brighton strain), monkeypox (Zaire strain), and vaccinia (Copenhagen strain) viruses are obtained from Centers for Disease Control and Prevention, Atlanta, Ga. The viruses are propagated in African green monkey kidney (Vero 76) cells. The Vero 76 and BALB/3T3 clone A31 cells (3T3 cells) used in the virus experiments are obtained from the American Type Culture Collection (Manassas, Va.). The cells are cultured in Dulbecco's high-glucose medium containing 10% fetal bovine serum. The serum concentration is reduced to 2% for assays and virus propagation. A low number of cell culture passages of poxviruses (passaged three times from originally obtained stocks) is used to initiate these studies.

Cells are plated in six-well plates which are infected with about 100 PFU of virus per well, the virus is adsorbed for 1.5 to 2 h, and then twofold dilutions of antiviral compounds are applied. The antiviral compounds may be from different classes of antiviral agents, such as those inhibiting viral DNA polymerases (cidofovir, cyclic HPMPC, and HPMPA), IMP dehydrogenase inhibitors (ribavirin and MPA), and C-c³ Ado, an inhibitor of S-adenosylhomocysteine hydrolase, and natural anti-virals such as Dragon's Blood. The incubation times for the viruses may be as follows: vaccinia virus, 3 days; cowpox virus, 4 days; monkeypox virus, 6 days; and camelpox virus, 7 days. At the end of the incubation plaque sizes are compared in the anti-viral treated wells. The cells are fixed and stained in 3% buffered formalin-0.2% crystal violet for 15 min and the plaques are counted.

To assay amount of virus surviving the anti-viral treatment, twelve-well plates of cells are infected with cowpox or monkeypox virus at about 100 PFU/well. After virus adsorption (1.5 to 2 h), the cells are fed maintenance medium with or without drug. Each day, a portion of the infected cells is frozen, thawed, and sonicated for 30 s. Subsequently, the medium (including both intracellular and extracellular virus produced during the infection) is titrated by plaque assay on new monolayers of Vero 76 cells. After 4 or 6 days, the cells are fixed and stained and the plaques counted.

A reduction in viral particles detected, or number of viral plaques counted as a result of Dragon's Blood treatment indicates that the Dragon's Blood composition is a potent anti-viral compound and may be useful in the treatment of pox virus infections, papilloma virus infections, and potentially for other viral infections, especially those affecting epithelial cells. Without intending to be limited to a particular theory of the invention, the Dragon's Blood compositions of the invention may have immunopotentiating activity in vivo against viruses that are distinct from direct antiviral activity measurable in aan in vitro assay. Consequently, a failure to redue viral plaques in vitro does not alone reflect lack of efficacy with respect to such virus in vivo.

The preceding examples can be repeated and have been repeated with similar results using the invention composition and invention method.

Without departing from the scope thereof, one skilled in the art can easily ascertain from the forgoing description the essential characteristics of this invention to adapt it to various uses and conditions.

The foregoing description and examples have been set forth merely to illustrate the invention and are not intended to be limiting. Because modifications of the disclosed embodiments incorporating the spirit and substance of the invention may occur to persons skilled in the art, the invention should be construed to include everything within the scope of the appended claims and equivalents thereof. The patents, patent application publications and other publications (e.g., Journal articles) referenced herein are incorporated in their entirety.

Although the applicant(s) invented the full scope of the claims appended hereto, the claims are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

1. A method of treating a Poxyiridae viral infection, comprising:
administering to a mammalian subject in need of treatment for a Poxyiridae viral infection a composition comprising sap from a tree of the genus Croton, in an amount effective to ameliorate symptoms of the infection.

2. A method according to claim 1, further comprising diagnosing a mammalian subject with a Poxviridae viral infection, wherein the diagnosed subject is selected for the administering step.

3. The method of claim 1, wherein the infection comprises a vertebrate Poxyzridae virus.

4. The method of claim 3, wherein the subject is human.

5. The method of claim 2, wherein the infection comprises at least one Poxviridae virus of a genus selected from the group consisting of Orthopoxvirus, Parapoxvirus, Avipoxvirus, Capripoxvirus, Leporipoxvirus, Suipoxvirus, Molluscipoxvirus and Yatapoxvirus.

6. The method of claim 5, wherein the genus is Orthopoxvirus and the virus is variola (small pox).

7. The method of claim 5, wherein the genus is Molluscipoxvirus and the virus is Molluscum contagiosum.

8-17. (canceled)

18. The method of claim 1, wherein the administration is topical.

19. The method of claim 1, wherein the composition further comprises at least one carrier selected from the group consisting of a cream, salve, foam, lotion, collagen preparation, gel, ointment, and combinations thereof.

20. The method of claim 1, wherein the administration step comprises applying a bandage to a Poxviridae lesion on the mammalian subject, wherein the bandage is coated or impregnated or contains the sap.

21. The method of claim 1, wherein the tree comprises a Croton species selected from the group consisting of salutaris, gossypifollus, palanostima, erythrochilus, lechleri, urucurana, xalapensis, draconoides, and hybrids thereof.

22. The method of claim 1 further comprising administering an anti-viral agent.

23. The method of claim 1 wherein the composition further comprises an anti-viral agent.
